(19) United States
(12) Patent Application Publication
(10) Pub. No.: US 2024/0255426 A1
(43) Pub. Date: Aug. 1, 2024

(54) Apparatus, Device and Method for Irradiating an in Particular Biological Specimen with a Holographic Optical Component

(71) Applicant: Robert Bosch GmbH, Stuttgart (DE)

(72) Inventors: Reinhold Fiess, Durbach (DE); Eugen Baumgart, Bad Teinach-Zavelstein (DE); Ingo Ramsteiner, Ettlingen (DE)

(21) Appl. No.: 18/551,686

(22) PCT Filed: Mar. 18, 2022
(86) PCT No.: PCT/EP2022/057119

§ 371 Date: Apr. 22, 2024

(30) Foreign Application Priority Data
Mar. 23, 2021 (DE) 10 2021 202 794.6
Mar. 17, 2022 (DE) 10 2022 202 634.9

(51) Int. Cl. G01N 21/64; C12Q 1/686; G02B 5/32

(52) U.S. Cl. CPC G01N 21/645; G01N 21/6486; G02B 5/32; C12Q 1/686; G01N 2021/6419; G01N 2021/6463; G01N 2201/0633; G01N 2201/0636

(57) ABSTRACT

An apparatus for irradiating an in particular biological sample, includes a first light source, a second light source, and at least one holographic optical component. The first light source, the second light source, and the holographic optical component are positioned relative to one another in such a way that first light from the first light source and second light from the second light source are deflected via the holographic optical component onto a common specimen region for irradiation of the specimen.

### PRIOR ART

In the biosciences in particular, numerous experimental methods rely on fluorescence phenomena. In addition to basic research, fluorescence measurements are also widely used in medical technology, for example in flow cytometry, in quantitative real-time PCR, in antibody testing, or in histopathology.

The light source is particularly important for the relevant instruments, because any fluorescence measurement requires excitation of the specimen with a selected wavelength band. The task of the light source is to provide light of high spectral density in the area of the absorbent band of the respective fluorophore. At the same time, however, the light must be limited to a precisely defined wavelength interval in order to avoid outshining the spectrally nearby fluorescence wavelengths. These requirements become more stringent when several dyes that have different, mostly overlapping excitation and fluorescent bands are to be addressed separately. The light source must then be able to switch between different excitation bands that are spectrally characterized by precisely defined wavelength bands having steep flanks.

According to the prior art, such light sources are realized either by providing a very wide-band light source (e.g., white light-emitting diode, incandescent lamp, or gas discharge lamp) with replaceable dielectric band-pass filters arranged on a slider or wheel, for example, or by using multiple light sources having either intrinsically restricted spectral range (laser, SLD) and/or greater bandwidth (e.g., colored LEDs), but each being spectrally defined by a fixed band-pass filter. When multiple light sources are used, the beam paths are typically made congruent by dichroic mirrors before the light strikes the specimen.

### DISCLOSURE OF THE INVENTION

### Advantages of the Invention

Based on the above background, the invention relates to an apparatus for irradiating a specimen. The apparatus comprises a first light source, a second light source, and at least one holographic optical component. The first light source, the second light source, and the holographic optical component are arranged in relation to one another in such a way that first light from the first light source and second light from the second light source are deflected via the holographic optical component onto a common specimen region for irradiation of the specimen.

The specimen preferably comprises a material which can be stimulated to fluorescence under suitable optical excitation, in particular due to fluorophores present in the specimen or added to the specimen. The specimen may in particular be a biological specimen. For example, the specimen can comprise components of a bodily fluid such as components of blood, urine, sputum, or a smear or tissue specimen from an animal or human. In particular, the specimen may include nucleic acids or portions of nucleic acids, preferably duplicated portions of nucleic acids from isothermal or polymerase chain reaction (“PCR”)-based duplication of nucleic acid portions. For example, the specimen may comprise a product from a detection method for the detection of pathogens, in particular a product from an isothermal or PCR-based DNA amplification, wherein the detection of the presence of certain pathogens is to be carried out in particular via a fluorescence-based selection of DNA specimens marked with fluorophores. In addition, however, the invention is generally suitable for any application and for any apparatus used for this purpose in which fluorescent test objects must be illuminated with multiple wavelength bands, in particular in the areas of life science, forensics, and in the protection and authenticity testing of products, in particular documents against product piracy.

The light sources may be, for example, light-emitting diodes (“LEDs”), superluminescence or laser diodes, incandescent lamps, gas discharge lamps, or light sources excited by primary sources, for example phosphorus-converted sources. Preferably, the first light source comprises a first emission spectrum different from a second emission spectrum of the second light source. Thus, the wavelength spectrum of the first light may preferably differ from that of the second light. In advantageous embodiments, the first or the second light source comprise beam-forming elements (e.g., concave mirrors, Fresnel or refractive lenses) and/or spectrally filtering elements (e.g., dielectric filters or colored glasses). In particular, the first or second light source may comprise a band-pass filter for limiting the emitted light in a defined manner. The spectral range of the light sources may advantageously be restricted by such band-pass filters in a more narrow band and with steeper edges than is possible solely by means of the intrinsic spectral width of the holographic optical component. Further, this may reduce scattered light having undesirable wavelengths. Alternatively or additionally, such beam-forming and/or spectrally filtering elements may be arranged in other locations of the apparatus, in particular between the holographic optical component and the specimen region or the specimen.

The holographic optical component (also abbreviated as “HOE”) is in particular an element having holographic properties that can be used for the optics of devices to replace, for example, conventional lenses, mirrors, and prisms. The HOE preferably comprises a substrate for mechanical stabilization (e.g., glass or plastic), as well as at least one or more optically active layers in which one or more holograms are inscribed. The HOE is configured, as noted above, to direct light of the first and second light sources at least partially onto the specimen region. The intrinsic wavelength selectivity of the holograms may preferably be used as a desired filter function in order to select a specific wavelength band for illumination of the specimen region and the specimen. In advantageous embodiments of the invention, the holographic optical component may comprise a reflection hologram or a transmission hologram configured as a surface hologram or preferably as a volume hologram. The holographic optical component is thus preferably configured to direct at least a portion of the light from the first light source and a portion of the light from the second light source to a predetermined specimen region for a specified location, relative to the HOE, of the first light source having the first emission spectrum and the second light source having the second emission spectrum.

The (common) specimen region means a particular spatial region relative to the HOE and preferably relative to the two light sources, in particular a particular spatial angle region in which a specimen can be placed for irradiating according to the invention.

The invention thus advantageously provides an excitation optic for fluorescence spectroscopy, wherein excitation light from different excitation channels may be directed via the holographic optical component to the specimen to be investigated. The holographic optical component may advantageously perform several functions, in particular due to holographic structures configured for this purpose in one or more holographic layers of the HOE. Firstly, the HOE is preferably configured to geometrically merge a plurality of beam paths, and thus to redirect light beams incident on the HOE from different directions into a common region, in particular a common direction, in particular via reflection or transmission caused by the HOE. In addition, the HOE may be configured to direct only light beams of certain wavelengths in a particular direction, preferably to the specimen. A further advantage is that band-pass filters can be omitted. Moreover, the HOE may be configured to form an intrinsic radiation distribution of one or more of the light sources, in particular to filter, diffract, focus/bundle, collimate, and/or widen without otherwise requiring typical filters, diffraction grids, lenses, or curved mirrors. Further, the HOE may be configured to diffract light of specified wavelengths, particularly at the short and/or long wave flank of a specified wavelength band. The HOE may preferably be configured to fulfill one or more of these functions simultaneously. In other words, the HOE can act as a beam shaper for light of specified wavelengths and/or from specified directions. Preferably, the HOE may also be permeable to light of other wavelengths and/or from other specified directions, i.e., affecting such light only slightly, if at all.

The invention thus advantageously allows the specimen to be illuminated with spectrally different light from the same direction via the HOE. In particular, wavelength-sensitive, deterministic redirection of light is possible by means of the invention, as described above. This is particularly advantageous when different fluorophores present in the specimen and having different excitation spectra are to be excited under the same geometric lighting conditions, i.e., when the specimen is to be exposed to the same illumination situation regardless of the spectral distribution of the light. In contrast to illumination of the specimen with spatially separated light sources, the same illumination made possible by the apparatus according to the invention has the particular advantage that inhomogeneities in the specimen are illuminated in the same manner by different excitation light and do not differently influence different fluorescence signals.

In contrast to the solution having a wide-band light source and interchangeable filters explained above, the apparatus according to the invention has the advantage that no movable parts and no associated mechanism susceptible to error and wear are required. Further, it is avoided that a large portion of the light generated by the broadband light source remains unused, that the light is screened by further measures, and that the associated waste heat must be dissipated. The invention also has particular advantages over the solutions having multiple light sources discussed above. The solution according to the invention can be implemented significantly more cost-effectively than the use of multiple light sources. This is because laser or superluminescence diodes are often needed for light sources having intrinsically restricted spectral ranges and comparatively expensive dielectric band-pass filters are needed for wide-band light sources. Moreover, due to the particular properties of the HOE, the apparatus according to the invention does not require any additional optical components, for example dichroic mirrors, for combining the beam paths of the different light sources.

The invention thus provides a resource-saving, compact, and comparatively inexpensive excitation optic, which is particularly suitable for use in low-cost molecular diagnostics, particularly for use in lab-on-a-chip platforms, preferably in quantitative real-time PCR with multiplex functionality, for example in point-of-care solutions.

In a preferred embodiment, the HOE and the two light sources are arranged relative to each other such that first light from the first light source and second light from the second light source are deflected via the HOE to a common light path or beam path for irradiation of the specimen, preferably via a reflection by the HOE, for which the HOE comprises a reflection hologram. The common light path or beam path can in particular be realized by at least partially overlapping, preferably completely overlapping beam paths or beam profiles. In the special case of planar waves of the light emitted by the light sources, the apparatus according to the invention allows an at least partially, preferably completely congruent wave front of the light beams deflected by the HOE.

In an advantageous refinement, the apparatus can also comprise more than two light sources as described above, wherein the further light sources can also comprise beam-forming and/or spectrally filtering elements as described above. According to advantageous embodiments, the apparatus comprises in particular between two and six, preferably between two and four light sources, in particular light sources suitable for molecular diagnostic applications. Thus, the specimen region may advantageously be illuminated in the same manner with many different light spectra.

According to a particular embodiment, the apparatus comprises at least one third light source and the holographic optical component is configured to be permeable to light of specified wavelengths from the third light source. A particularly compact design of the apparatus is thus advantageously possible, in which the HOE is located between the third light source and the two other light sources as well as the specimen. In other words, the HOE is configured to deflect light from the first and the second light sources to the specimen and to allow light from the third light source to pass through, so that light from all three light sources preferably strikes the specimen region and in particular the specimen from the same direction. According to the present embodiment, the HOE thus preferably has no significant effect on light of specified wavelengths from the third light source, with the exception of not completely avoidable effects due to interfacial and material properties (Fresnel reflection and slight scattering and absorption). In preferred embodiments, further light sources may be placed on the same side of the HOE as the first and second light source or on the same side as the third light source, such that selected light from these light sources is also deflected via the HOE or directed through the HOE. In other embodiments, the HOE may be configured to allow light of predetermined wavelengths to pass through the HOE from a specified direction, thereby altering the direction, such that an effect similar to diffraction of light occurs.

In a particularly advantageous further development of the invention, the apparatus comprises one or more further optical elements, in particular apertures, lenses, and/or shutters or other beam-shaping and/or spectral filtering elements as described above. These further optical elements may be arranged with respect to the holographic optical component in order to change, in particular to bundle, focus, and/or redirect, light from the light sources deflected by the HOE. This has the advantage that the light deflected by the holographic optical component can be adjusted further before striking a specimen.

In a particularly preferred refinement of the invention, the first light source and the second light source are disposed on a surface of a cone or a truncated cone, in particular on a circle about the exit axis of the holographic optical component. In particular, the exit axis means the direction in which the HOE emits or diverts light as intended due to its design. The holographic optical component may preferably be arranged at or in the tip of the cone or the truncated cone. The surface may mean only the theoretical geometrical shape of the geometric object of a cone or a truncated cone. Alternatively, the first and/or the second light source may actually be arranged on or in a surface of a preferably at least partially conical, pyramidal, frustoconical, or frustopyramidal component. This has the advantage that rotationally symmetrical molded parts can be manufactured inexpensively and precisely and that the cost of adjustment is significantly reduced due to the utilization of the geometry of the molded part. In a preferred boundary case, the first light source and the second light source are arranged on a circle about the exit axis of the holographic optical component. In particular, this component may further be a hollow body. In this case, the light sources and/or the HOE may be mounted internally, in particular on or in an inner wall of the hollow body.

According to a further advantageous embodiment, the apparatus comprises one or more mirrors. The mirrors are arranged with respect to the light sources and the holographic optical component such that light emitted by the light sources is deflected via the mirrors to the holographic optical component. Preferably, the mirrors may be disposed at or on the surface of the component described above. If the apparatus comprises a hollow body as described above, one or more mirrors may be mounted on an inner side of the hollow body, in particular on an inner side of the surface of the hollow body. A portion of the surface may also be implemented as a mirror, for example due to a reflective area of a surface of the shell, for example a reflective metallic surface. Thus, the one or more mirrors may be part of a tapering surface, in particular part of a surface of a cone, truncated cone, pyramid or truncated pyramid, preferably configured as a hollow body.

In a further advantageous embodiment, the apparatus comprises a transparent body arranged such that light from at least one light source may be passed through the transparent body, preferably by taking advantage of total reflection within the body. This has the advantage that light beams from at least one light source can be directed to the HOE via the solid transparent body and/or directed by the HOE to the specimen region. In particular, the transparent body may be arranged between at least one light source, preferably between both light sources, and the HOE. To this end, the transparent body can be made at least partially of glass or transparent plastic. According to an advantageous embodiment, the HOE can be mounted or disposed on or in the transparent body. This has the advantage that no separate substrate is needed for the placement of the HOE. In a preferred embodiment, at least one light source and the HOE are adjacent to the transparent body, wherein the body may be formed as a plate. This has the advantage that light coupled into the body by the light source can be passed through the body to the HOE, preferably by taking advantage of total reflection. For example, the HOE can comprise a reflection hologram or a transmission hologram for coupling out the light from the body.

In a further advantageous embodiment of the invention, the light sources are disposed on a common board. This has the advantage that only one component is required for a well-defined placement of the light sources.

According to a particularly advantageous refinement of the invention, the holographic optical component is configured to collimate or focus incident radiation. Incident radiation may be a planar wave, diverging radiation, or converging radiation. The diverging radiation may in particular be part of radiation having a spherical wave front, such as is transmitted from an approximately punctiform light source. A further object of the invention is a device for the investigation of a particularly biological specimen, wherein the device comprises an apparatus according to the invention and a measuring device for the detection of light emitted from a received specimen, in particular fluorescent light.

A further object of the invention is a method for irradiating an in particular biological specimen by means of the apparatus according to the invention, that is, a method wherein first light from a first light source and second light from a second light source are deflected via a holographic optical component onto a common specimen region, in particular on a common light path, to irradiate the specimen. As explained above, the method according to the invention is particularly suitable for application in lab-on-a-chip platforms, preferably for fluorescence measurements for (quantitative) real-time PCR with multiplex functionality, for example for point-of-care solutions. Regarding the further advantages of the method according to the invention, we refer to the advantages of the apparatus according to the invention described above.

### BRIEF DESCRIPTION OF THE DRAWINGS

Exemplary embodiments of the invention are shown schematically in the drawings and explained in more detail in the following description. The same reference signs are used for the elements having the same effect and shown in the various drawings, so repeated description of these elements is omitted.

Shown are:

FIGS. 1 to 6 Exemplary embodiments of the apparatus and device according to the invention, and

FIG. 7 A flow diagram of an exemplary embodiment of the method according to the invention.

### EMBODIMENTS OF THE INVENTION

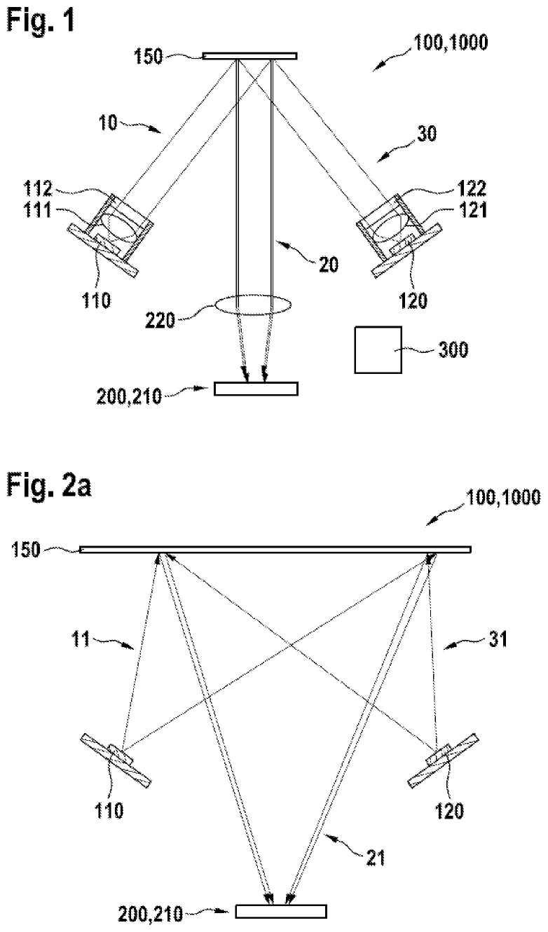

FIG. 1 thus also shows a first exemplary embodiment of the apparatus 100 according to the invention. The apparatus 100 comprises a holographic optical component 150 (“HOE”), a first light source 110, and a second light source 120 to illuminate a specimen 210 placed in a specimen region 200. For example, as described above, the specimen comprises DNA segments multiplied via polymerase chain reaction, which are to be detected via excitation of fluorescent probes in the specimen. FIG. 1 also shows an exemplary embodiment of the device 1000 according to the invention comprising the exemplary embodiment of the apparatus 100 according to the invention and a measuring device 300, wherein the measuring device 300 is configured, for example as a camera, to capture fluorescent light emitted by the specimen 210.

The HOE 150 comprises one or more holograms, for example as volume holograms, which may be embedded in one or more photopolymer layers. The photopolymer layers may be applied to a suitable carrier, for example a glass plate, or may also be embedded between two carriers, for example in laminated form. In one embodiment as a volume hologram, the hologram may be imprinted into the layer(s) according to the desired arrangement of the light sources towards the HOE and specimen region. In an embodiment as a hologram having local variation of the layer thickness, the hologram can be embossed into the layer, for example, using an embossing template (also called “master”).

As shown in FIG. 1, the first light source 110, the second light source 120, and the holographic optical component 150 are arranged in relation to one another in such a way that first light 10 from the first light source 110 and second light 30 from the second light source 120 are deflected via the holographic optical component 150 onto a common specimen region 200 for irradiation of the specimen 210. To this end, the HOE 100 is configured to direct planar light waves, particularly collimated radiation, of a first wavelength from a first direction onto a common specimen region 200. Under “light waves of a . . . wavelength” herein and in the following, in particular light wavelengths having wavelengths from a wavelength interval of finite width are meant, i.e., in particular light wavelengths having wavelengths from a particular wavelength spectrum of finite width.

In this example, the HOE 150 is specified to deflect planar light waves (collimated beams) of a first wavelength from a first direction 10 to a second direction 20, preferably perpendicular to the surface of the HOE 150 as shown in FIG. 1. The HOE 150 is further configured so that planar light waves of a second wavelength from a third direction 30 are also redirected in the second direction 20, i.e., both light paths are combined. A reflection hologram is preferably used as shown, because in practice better quality in terms of diffraction efficiency and light scattering can thereby be achieved. Alternatively, however, a configuration of the apparatus 100 having a transmission hologram is also possible. As explained above, the HOE 150 can in particular (in addition) be configured to perform wave selection depending on the direction of the light incident on the HOE 150.

As shown in FIG. 1, two light sources 110, 120, preferably light-emitting diodes (LEDs), are mounted so as to be able to illuminate the HOE 150, wherein the wavelength and incident direction must correspond to the design of the hologram of the HOE 150. For example, the first light source 110 may be an LED radiating light having a central wavelength of about 472 nanometers (nm) and a half-width of 15 nm. For example, the second light source 120 may also be an LED radiating light having a central wavelength of about 530 nm and a half-width of 32 nm. Thus, in the present example, the HOE 150 is configured to redirect light coming from the first light source 110 at a wavelength of 472 nm and light coming from the second light source 120 at a wavelength of 530 nm to a common direction 20 towards the specimen region 200. The efficiency of the hologram of the HOE 150 typically follows a Lorentz distribution having a width of 15 nm around the respective central wavelength, for example, which does not necessarily have to exactly match the central wavelengths of the LEDs 110, 120. Preferably, the HOE 150 restricts the deflected radiation to the range given by its own half-width and keeps it stable depending on operation, in particular depending on temperature and energizing, and despite the central wavelengths of the LEDs varying by manufacturing tolerances.

The HOE 150 thus combines the two incident beams from different directions 10, 30 onto a common beam path 20, so that the specimen 210 can be illuminated by switching on and off the individual light sources 110, 120 having different wavelengths from the same direction 20. The exemplary embodiment shown in FIG. 1 for two light sources 110, 120 and two directions 10, 30 having two different wavelength spectra may be extended with additional light sources as needed. Further, the symmetry of the light sources 110, 120 with respect to the HOE 150 shown in FIG. 1 is not mandatory. In particular, the light sources 110, 120, unlike as shown, may be disposed with respect to the HOE such that light emitted therefrom forms different incident angles to a surface normal of the HOE 150. The HOE 150 can also be configured to redirect the incident light from the light sources 110, 120 not along the surface normal of the HOE 150, as shown, but at a different angle to the surface normal.

For molecular diagnostic applications, such as in a quantitative polymerase chain reaction with multiplexing, the apparatus 100 may preferably comprise two to six light sources, particularly preferably four light sources. For example, the two or more light sources may be placed on a circle about an axis perpendicular to the HOE, in particular about a surface normal of the HOE that lies in the second direction 20 according to FIG. 1, as explained further below. This has optical, design, and manufacturing advantages. The maximum number of light sources is limited only by practical reasons, in particular by limited installation space and a decreasing efficiency of the HOE 150 with increasing degree of multiplexing.

For an improvement of the light yield, it may be advantageous to concentrate the light of the light sources 110, 120 onto the HOE 150 by means of optical collecting elements 111, 121 by collimating or bundling, depending on the optical design of the hologram of the HOE 150 and beam path. Refractive or diffractive optical elements known in the art may be employed here, for example, simple lenses, parabolic collecting lenses, Fresnel lenses, HOEs, or combinations of such elements.

Although the hologram of the HOE 150 is fundamentally wavelength selective, the apparatus 100 may have band-pass filters 112, 122, as shown in FIG. 1. Preferably, as shown, these are arranged downstream of the collecting members 111, 121, where the beams emitted by the light sources 110, 120 have as narrow an angle distribution as possible. Preferably, the space of the light source 110, 120 and the collecting optics 111, 121 is visually separated from the space of the HOE 150 (for example, by screening surfaces, or in a tube as shown in FIG. 1) by means of the band-pass filters 112, 122, so that no light fractions from the light source 110, 120 to the HOE 150 can bypass the band-pass filter 112, 122. The optional band-pass filters 112, 122, as described above, have the advantageous effect of restricting the spectral range of the light sources 110, 120 in a more narrow band and with steeper edges than is generally possible due to the intrinsic spectral width of the hologram of the HOE 150. Further, this may reduce scattered light having undesirable wavelengths.

As further shown in FIG. 1, the apparatus 100 may comprise a further collecting element 220, in particular a collecting lens, for focusing the beams 20 on the specimen 210. In addition, the apparatus 100 may include further optical elements such as apertures, lenses, and shutters to further shape the merged beam path 20 after the HOE.

The exemplary embodiment shown in FIG. 1 is particularly suitable for redirecting planar waves or collimated beams. This is particularly advantageous when interference filters 112, 122 (band-pass or edge filters) are to be used in addition to cleaning scattered light, for example before an individual light source or a multiband-pass filter downstream of the HOE 150.

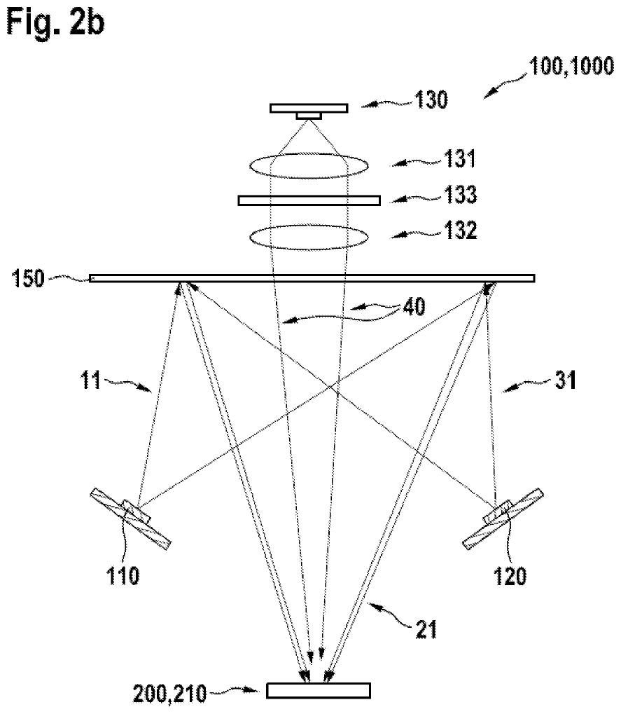

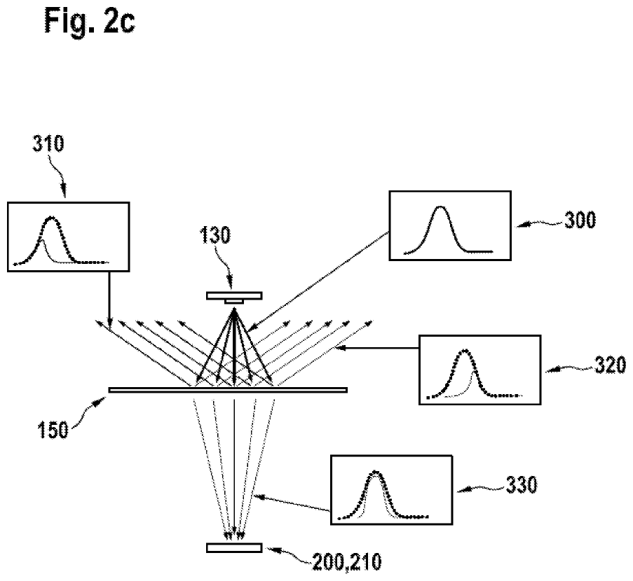

However, the apparatus 100 according to the invention also allows the HOE 150 to not only realize a wavelength-selective deflection, but also to shape the wave front of the incident light. FIG. 2a shows an exemplary embodiment in which the spherical wave 11, 31 emanating from the respective light source 110, 120 is deflected into a spherical wave 21 extending towards the specimen 210. Formulated as a beam pattern, the divergent light of the light sources 110, 120 is thereby collected by the HOE 150 over a large cross-sectional area and focused on the specimen 210. Alternatively, exemplary embodiments of the apparatus 100 may also be implemented, in which the HOE 150 provides a comparatively flat wave on either the specimen or light source side, and enables the integration of a band-pass filter on this side, but on the other side acts to collect or bundle. As indicated in FIG. 1 above, the symmetry of the light sources 110, 120 with respect to the HOE 150 shown in FIG. 2 is also not mandatory.

FIG. 2b shows a further development of the exemplary embodiment of FIG. 2a, which comprises an additional third light source 130 to the first two light sources 110, 120, wherein light from the third light source 130 preferably irradiates the specimen without interference by the HOE. In particular, the third light source 130 may be disposed on the side of the HOE 150 facing away from the first two light sources 110, 120. The HOE 150 may preferably be configured to be permeable to light 40 of a specified wavelength range from the third light source 130. This has the advantage that such light from the third light source 130 can additionally act on the specimen 210 practically without interference, preferably from the same direction as the light from the first and second light source 110, 120 deflected via the HOE. The advantage of the high wavelength selectivity of HOEs is used in order to realize a reflection of light and a transmission of other light at the same time, so that light sources 110, 120, 130 can advantageously be placed at different locations with respect to the HOE and irradiation of the specimen 210 by means of light from all of these light sources 110, 120, 130 from the same direction is still possible. In other words, the HOE 150 may be configured to divert light of particular wavelengths from particular directions to a desired target direction while allowing light from particular other wavelengths and/or from particular other directions to pass virtually unhindered in the same target direction. For example, the light sources 110, 120, 130 could have peak wavelengths of 470 nm, 530 nm, 590 nm, 630 nm, 405 nm, or 385 nm. If the holographic material is not suitable for one of the wavelengths desired for the application, for example for 385 nm, it is possible to provide the source 130 having the same wavelength according to FIG. 2b. The first exemplary embodiment according to FIG. 1 can also be further refined with respect to the HOE 150 and the third light source 130 accordingly. As shown in FIG. 2b, the apparatus 100 may include further lenses 131, 132, and/or band-pass filters 133 disposed upstream of the third light source 130 to collect/dissipate/filter light from the third light source as needed.

Alternatively, the HOE 150 may be configured to shape, in particular to filter, bundle, diffuse, and/or diffract light from the third light source 130, and in particular embodiments as already stated above, light of pre-determined wavelengths from the first and/or second light source 110, 120. This has the advantage that the HOE performs the functions of filters, lenses, and diffraction gratings. FIG. 2c illustrates, by way of example, beam forming by the HOE 150 of light emitted by the third light source 130. According to the present example, the HOE 150 is configured to diffract a specified wavelength spectrum 300 at the short wave edge 310 and/or at the long wave edge 320 (and thus to effectively trim the spectrum 300) and/or to bundle light 330 passing through the HOE 150, similar to a collecting lens. Thus, an intrinsic spectrum of the light source 130 can be effectively trimmed in a narrower shape, preferably having steeper edges, advantageously resulting in a spectrum incident on the specimen 210 which is more robust against fluctuations in temperature, operating current, or component tolerance of the light source 130. In a preferred configuration, the HOE may also be configured to transfer the diffracted light fractions 310, 320 into an internal mode and from there preferably into an absorber material or beam trap to avoid stray light.

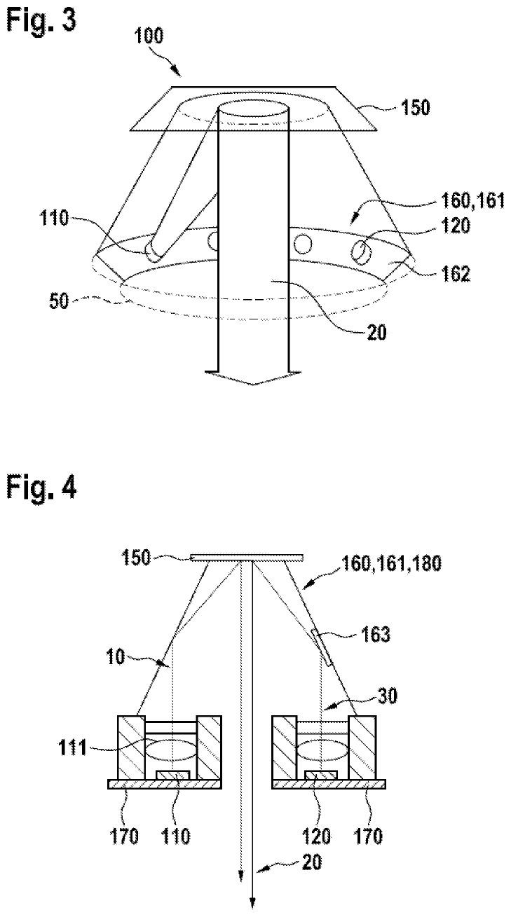

As already mentioned above, it may be advantageous for manufacturing to provide the location of the light sources 110, 120 on a circle 50 about the exit axis 20. In the exemplary embodiment shown in FIG. 3, multiple light sources 110, 120 are disposed on the surface 161 of a truncated cone 160 such that the optical axes thereof face the center of the HOE 150 without further adjustment. In particular, the light sources 110, 120 are disposed along a circle 162 about the exit axis 20 of the HOE 150. The truncated cone 160 is preferably arranged rotationally symmetrically about the exit axis 20 of the HOE 150, wherein the exit axis is perpendicular to the laminar HOE 150, i.e., is congruent with a surface normal of the HOE 150.

In the exemplary embodiment shown in FIG. 4, the first light source 110 and the second light source 120 are arranged on a common board 170, which is technically advantageous. The board 170 can in particular be a substrate typical for optical components, for example a printed circuit board that is typical in electronics. For a high power consumption of the light sources 110, 120, the board may comprise a core of thermally conductive material for dissipating the waste heat, for example comprising aluminum. The directing of the collimated beams 10, 30 on the HOE 150 can then be accomplished as shown via an at least partially mirrored surface on the inside of a truncated pyramid 160 or truncated cone 160, or alternatively via a mirror 163 mounted on the inside. A mirroring of the inner surface of the truncated shape 160 may be realized, for example, by reflective metal surfaces, particularly by vapor-deposited aluminum or silver. The reflective surfaces could also be domed and thus also perform an optical function. As also indicated in FIG. 3, the HOE 150 can also be disposed on or in the truncated shape 160.

Instead of a hollow body 160, the apparatus 100 can also have a solid, transparent body 180 in which the beam redirection is performed via total reflection. To this end, the body 180 may comprise glass or transparent plastic.

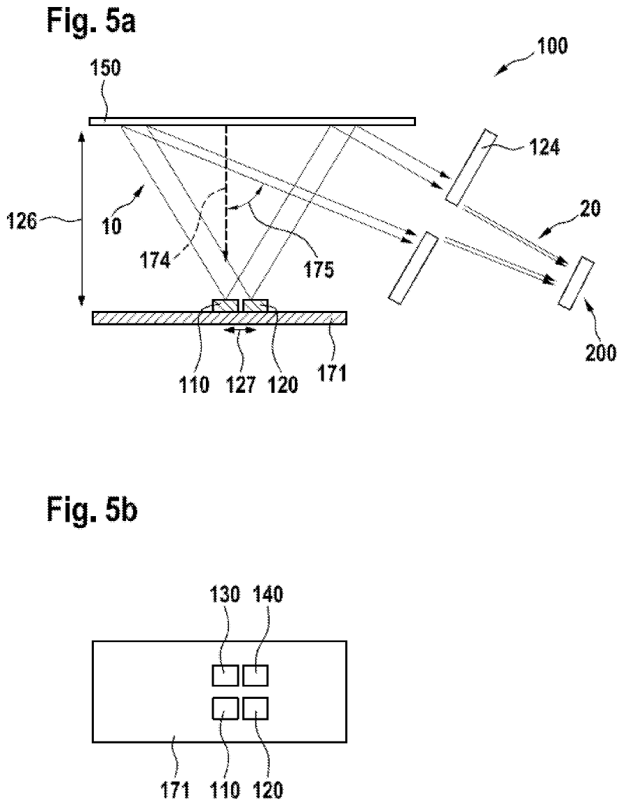

FIG. 5a shows a further exemplary embodiment of the apparatus 100 according to the invention comprising a plurality of light sources, in particular light-emitting diodes, in the present example four light sources, 110, 120, 130, 140. The LEDs 110, 120, 130, 140 are arranged on a board 171, for example in a two-by-two grid arrangement as shown in the top view of the board 171 in FIG. 5b. Alternatively, the apparatus 100 may also comprise more or fewer light sources, in particular disposed at minimal distance from one another, as described below.

The HOE 150 is arranged opposite a surface of the board 171, which is preferably configured to redirect perpendicularly incident, and in the present example approximately spherical, light 10 due to the approximately point-shaped light sources 110, 120, 130, 140 into a common specimen region, in particular in a common direction 20, at an angle 175 to the surface normal 174 of the HOE 150. For example, the angle 175 may have a value of between 40 and 70 degrees, preferably between 50 and 50 degrees. Preferably, the angle should be chosen to avoid Fresnel reflections, which occur particularly when the angle of incidence on the hologram corresponds to the angle of reflection. For a largely congruent illumination of the HOE 150, a distance 126 between the board 171 and HOE 150 is preferably at least three times greater, particularly preferably at least five times greater than a distance 127 between the light sources 110, 120, 130, 140. Therefore, it is also advantageous if the light sources 110, 120, 130, 140 are placed as close to one another as possible in order to achieve a good overlap of the emission cones.

As shown, the apparatus 100 can also have an aperture 124 to bring light deflected from the HOE 150 onto a congruent region as much as possible and thus to realize an illumination situation of the specimen region 200 that is as identical as possible for all light sources.

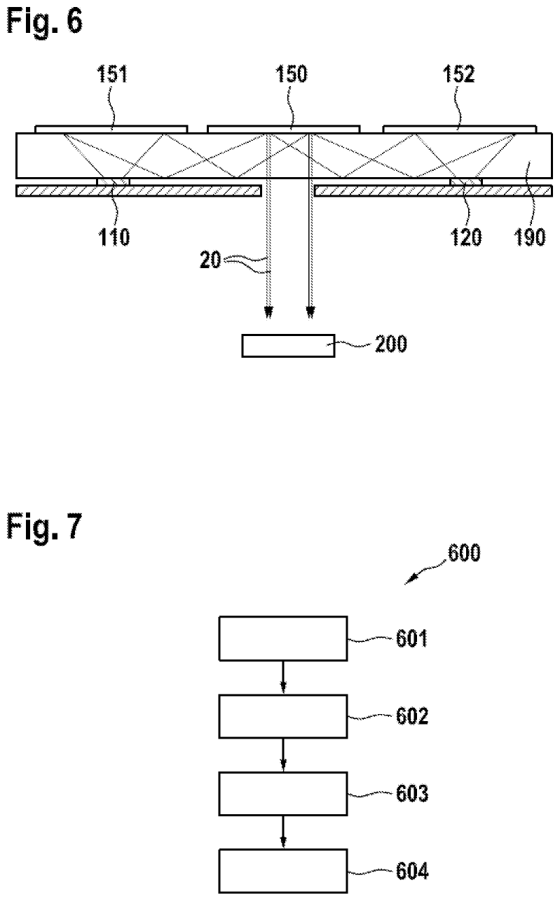

Particularly compact and minimalistic hardware concepts are possible if one allows the light beams to be propagated in the carrier medium of the hologram or the HOE, usually a glass plate, instead of an open beam arrangement. Such an arrangement is shown in a further exemplary embodiment of the apparatus 100 according to the invention according to FIG. 6. In the present example, the apparatus 100 comprises a transparent plate 190 as a transparent body 180 to which the two light sources 110, 120 in the form of LEDs are adjacent as much as possible without air gaps (possibly imparted by a layer having an adjusted refractive index). For example, the transparent plate 190 may be a glass plate 190 or other transparent carrier medium, for example based on transparent plastic. The light coupled by the light sources 110, 120 to the transparent plate 190 is deflected to the common specimen region 200 by a HOE 150, also comprising a reflection hologram, adjacent the plate 190. In addition to taking advantage of total reflection within the plate 190, the apparatus may include further HOE 151, 152 which also abut the plate and deflect incident radiation, preferably towards the first HOE 150. As shown in FIG. 5, all three HOE 150, 151, 152 are technically advantageously located on the same side of the plate 190 for manufacturing and each comprises a reflection hologram. Thus, the light beams fed into the plate 190 by the light sources 110, 120 are directed to the first, intervening HOE 150 both via total reflection and via the further HOE 151, 152 that couples the light 20 exiting from the plate 190. Alternatively, the HOE 150 could also have a transmission hologram and/or be disposed on the opposite side of the plate 190. Alternatively, the first HOE may also extend over a greater width along the plate 190, thus replacing the further HOE 151, 152.

FIG. 7 shows a flow diagram 600 for an exemplary embodiment of the method 600 according to the invention, which can be performed, for example, by means of one of the exemplary embodiments according to FIGS. 1 to 5. In a first step 601 of method 600, the apparatus 100 according to the invention is provided. In a second step 602, a specimen 210 may be placed in the common specimen region 200. In a third step 603, light from the first light source 110 and thereafter or simultaneously, light from the second light source 120 is deflected via the HOE to the common specimen region, for example to excite fluorophores in the specimen 210. In a fourth step 604, which can be performed simultaneously with the third step 603, the fluorescence radiation emitted by the specimen 210 is acquired by means of the measuring device 300, for example a camera 300 or a photodiode, for a subsequent analysis of the specimen 210.

1. An apparatus for irradiating a biological specimen, comprising:
a first light source;
a second light source; and
at least one holographic optical component,
wherein the first light source, the second light source, and the holographic optical component are positioned relative to one another in such a way that first light from the first light source and second light from the second light source are deflected via the holographic optical component onto a specimen region for irradiation of the specimen.

2. The apparatus according to claim 1, wherein the first light and the second light comprise different wavelength spectra.

3. The apparatus according to claim 1, further comprising:
a third light source,
wherein the holographic optical component is permeable to third light from the third light source.

4. The apparatus according to claim 1, further comprising:
one or more optical elements configured to bundle and/or focus light from the first and second light sources diverted by the holographic optical component.

5. The apparatus according to claim 1, wherein at least one of the first light source and the second light source is arranged on a surface of a component.

6. The apparatus according to claim 1, wherein at least one of the first light source and the second light source is arranged along a circle about an exit axis of the holographic optical component.

7. The apparatus according to claim 1, further comprising:
one or more mirrors arranged with respect to the first and second light sources and the holographic optical component such that the first and second light emitted by the first and second light sources is directed via the mirrors to the holographic optical component.

8. The apparatus according to claim 7, wherein the one or more mirrors are part of a tapering surface of a component.

9. The apparatus according to claim 1, further comprising:
a transparent body arranged such that at least one of the first light and the second light is passed through the transparent body.

10. The apparatus according to claim 9, wherein at least one of the first light source and the second light source and the holographic optical component are adjacent the transparent body.

11. The apparatus according to claim 1, wherein the first and second light sources are disposed on a common board.

12. The apparatus according to claim 1, wherein the holographic optical component is configured to collimate or focus incident radiation.

13. A device for investigating a biological specimen comprising:
an apparatus for irradiating a specimen, comprising:
a first light source;
a second light source; and
at least one holographic optical component,
wherein the first light source, the second light source, and the holographic optical component are positioned relative to one another in such a way that first light from the first light source and second light from the second light source are deflected via the holographic optical component onto a specimen region for irradiation of the specimen; and
a measuring device configured to detect light emitted from the specimen.

14. A method for irradiating a biological specimen, comprising:
deflecting first light from a first light source and second light from a second light source via a holographic optical component onto a common specimen region for irradiation of the specimen.

15. The method according to claim 14, wherein the method is employed in a polymerase chain reaction.

16. The apparatus according to claim 5, wherein the component is a cone, a truncated cone, a pyramid, or a truncated pyramid.

17. The apparatus according to claim 8, wherein the component is a cone, a truncated cone, a pyramid, or a truncated pyramid.

18. The apparatus according to claim 1, wherein the holographic optical component is configured to collimate or focus diverging light having a spherical wavefront.

19. The device according to claim 13, wherein the measuring device is configured to detect light fluorescent emitted from the specimen.

20. The method according to claim 14, wherein the method is employed in a real-time quantitative polymerase chain reaction.
